(19) United States
(12) Patent Application Publication
(10) Pub. No.: US 2005/0113677 A1
(43) Pub. Date: May 26, 2005

(54) Apparatus and method for registering the position of a surgical robot

(76) Inventors: Brian Davies, London (GB); Simon Harris, London (GB); Matjaz Jakopec, London (GB); Ferdinando Rodriguez Y Baena, London (GB)

Correspondence Address: WALLENSTEIN WAGNER & ROCKEY, LTD, 311 SOUTH WACKER DRIVE, 53RD FLOOR, CHICAGO, IL, 60606, US

(21) Appl. No.: 10/495,896

(22) PCT Filed: Nov. 1, 2002
(86) PCT No.: PCT/GB02/04945

(30) Foreign Application Priority Data
Nov. 19, 2001 (GB) 0127659.1

(51) Int. Cl. A61B 19/00

(52) U.S. Cl. 600/424; 606/130

(57) ABSTRACT

An apparatus and method for the position of a surgical robot, prior to its use for computer-aided surgery, has a probe (37) on the end of a robot, allowing the location of the robot to be registered with respect to a bone (33) to be cut. Prior to the procedure, the location of the robot is also registered with respect to a bone clamp (10) by touching the end of the probe (37) into depressions (15) formed in the clamp surface. If the robot needs to be moved, with respect to the bone, its position can be accurately re-registered by touching the probe tip back into some of the depressions (15) in the clamp. The system allows easy re-registration without the need either for expensive tracking systems or the use of fiducial markers inserted into the patient's bone.

The invention relates to an apparatus and method for registering the position of a surgical robot. It is particularly although not exclusively applicable to the registration of robots used to carry out orthopaedic procedures.

In recent years, robotic systems for assisting in medical procedures, including surgery, have become increasingly common. Typically, a pre-operative image is taken of the area of the patient to be operated upon (for example using CT—Computer Tomography Data), with the robot being used during surgery to guide the cutting or other surgical instruments on the basis of the pre-operative image. To ensure that there is an accurate alignment between the image and the three-dimensional “real life” position of the patient, an initial registration procedure is carried out.

One way of achieving accurate registration is to image not only that part of the patient to be operated on (for example a bone) but also some metallic fiducial markers that have, prior to the procedure, been screwed in or otherwise attached to the bone. Registration then consists of aligning the known position of the actual markers on the patient with the corresponding virtual positions of those markers within the computer, as recorded in the CT image. An alternative approach, which does away with the use of fiducial markers, is disclosed in U.S. Pat. No. 6,033,415: this relies on registering certain identifiable points on a patient's long bone with corresponding points as recorded on a digital image of the same bone. In either approach, prior to commencement of surgery, the surgeon uses a movable locating arm or probe on the robot to “learn” the position of the bone or of the fiducial markers; the registration procedure is then automatically carried out, for example by making a least squares fit based upon the known positions which have been touched by the locating arm and the corresponding positions within the digitised image.

Robotic or CAS (Computer Aided Surgery) systems currently used by the applicant make use of a relatively small robot which is held in an appropriate position for surgery by means of a gross positioning system. Once the robot has been correctly positioned with respect to the patient, the gross positioner is locked into place and is not moved during the surgical procedure. The robot itself—held on the end of an arm of the gross positioner—has a certain number of degrees of freedom allowing it to be moved on the gross positioner arm to reach the required surgical area. Location sensors on the robot ensure that the robot location is always known with respect to the gross positioner, so surgery can proceed unhindered provided that there is no relative movement between the patient and the gross positioner.

Unfortunately, it is not always convenient or even possible for a surgeon to be able to carry out a lengthy series of procedures without moving the patient or the gross positioner. The surgeon may need to move the patient in order to reach a difficult-to-access area, or the gross positioner (and hence the robot) may need to be removed in order to allow access for another (non-robotic) procedure to be undertaken. Whatever the reason, if the robot then needs to be put back and further robotic procedures undertaken, a re-registration procedure has to be used to ensure that the computer image is once again properly aligned with the new three-dimensional position of the patient.

One approach to re-registration is to provide the gross positioner with a system allowing for the internal measurement of joint angles. From a knowledge of the joint angles, and of the positioner geometry, the exact original placement of the robot can be determined. When the robot is put back, the internal angles are re-measured, and the new position of the robot can be calculated. Such an approach is, however, not entirely convenient: it requires that the gross positioner be equipped for such a task, which is expensive, and in some cases (such as where ball jointed passive positioning systems are in use) measuring individual joint angles is difficult.

An alternative approach is to use an external co-ordinate measurement system, for example an optical tracking system, to locate in three dimensions the position of the gross positioner and/or the robot. Both the original position of the gross positioner and/or robot is measured, as is the new position, allowing an appropriate mapping to be made from one reference frame to the other. While such an approach is effective, it is also expensive as it requires the use of a separate, accurate, co-ordinate measurement system in addition to the gross positioner and the robot itself.

A third option is to re-register the robot, after it has been replaced, either with the patient's bone or with fiducial markers secured to the bone. In many surgical situations, re-registration with the bone itself may be impossible, because the surfaces used for registration will have been removed during surgery. Even if those surfaces are still present, registration with the bone itself is time consuming since a large amount of surface data needs to be processed to achieve accurate re-registration. Re-registration to fiducial markers avoids these difficulties, but of course requires that the markers themselves were put into place before the pre-operative scan was made. The use of these markers requires that the patient undergo a separate procedure to have them inserted. That separate procedure itself takes time, increases the risk of infection, and may be painful and inconvenient for the patient.

It is an object of the present invention at least to alleviate these difficulties of the prior art.

It is a further object of the invention to provide an inexpensive and easy to use apparatus and method for positioning a surgical robot.

It is a further object of the invention to provide an easy to use and inexpensive apparatus and method for re-registering a surgical robot to a patient (for example to a bone of the patient) after the robot has been moved relative to the patient at the end of an earlier surgical procedure.

It is a further object of the invention to provide an apparatus and method for positioning a surgical robot that does not require the use of fiducial markers.

According to a first aspect of the present invention there is provided apparatus for registering the position of a surgical robot prior to undertaking a surgical procedure, comprising a patient restraint including a plurality of marker locations thereon for receipt of a probe associated with a robot the position of which is to be registered.

According to a further aspect there is provided a system for registering the position of a surgical robot prior to undertaking a surgical procedure, the system comprising:
(a) a surgical robot;
(b) a probe associated with the robot; and
(c) a patient restraint including a plurality of marker locations thereon for receipt of the probe.

According to yet a further aspect there is provided a method of registering the position of a surgical robot prior to undertaking a surgical procedure, comprising:
(a) clamping a body part in a fixed position by means of a patient restraint; and
(b) registering the robot position by touching a probe associated with the robot to a plurality of marker locations on the patient restraint.

The robot mentioned above may either be an active robot or, alternatively, a passive constraint robot.

In a preferred embodiment of the method, the following steps are involved.

1. Register the robot to the patient's bone by touching exposed surfaces of the bone with a probe;

2. Register the robot to a clamp or to a bone restraint system which is secured to the bone;

3. After any required first surgical procedure has been completed, move the robot relative to the patient, or vice versa; and

4. Re-register the robot in its new position to the clamp or to the patient restraint, ready for a second surgical procedure to take place.

The apparatus, system and method of the present invention allows accurate and precise re-registration of a surgical robot, after it has been moved, without the need to use either fiducial markers which have been surgically implanted into a patient's bone, or an expensive surgical navigational tracking system.

The invention may be carried into practice in a number of ways and two specific embodiments will now be described, by way of example, with reference to the accompanying drawings, in which:

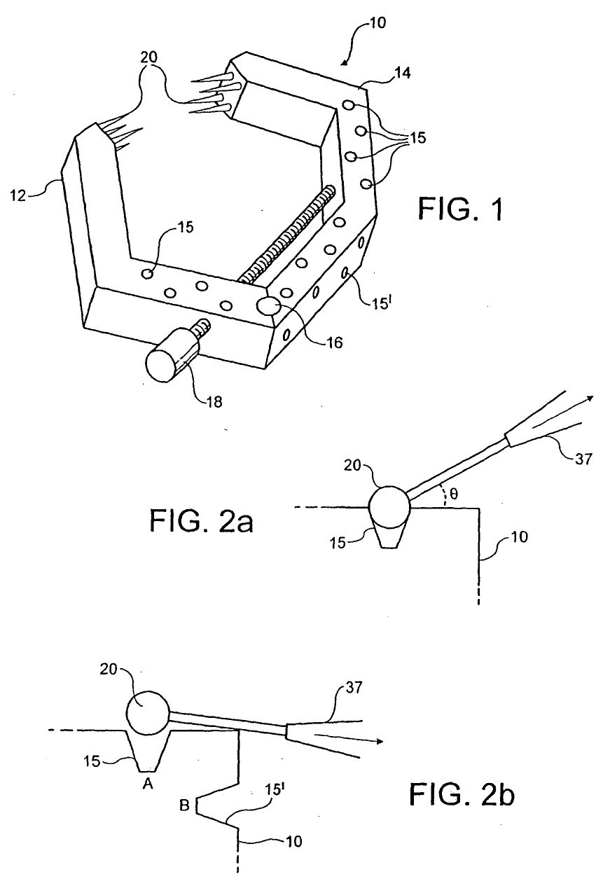

FIG. 1 shows a bone clamp according to a first embodiment of the invention;

FIGS. 2a and 2b show, respectively, correct and incorrect methods for registering a robot to the clamp of FIG. 1;

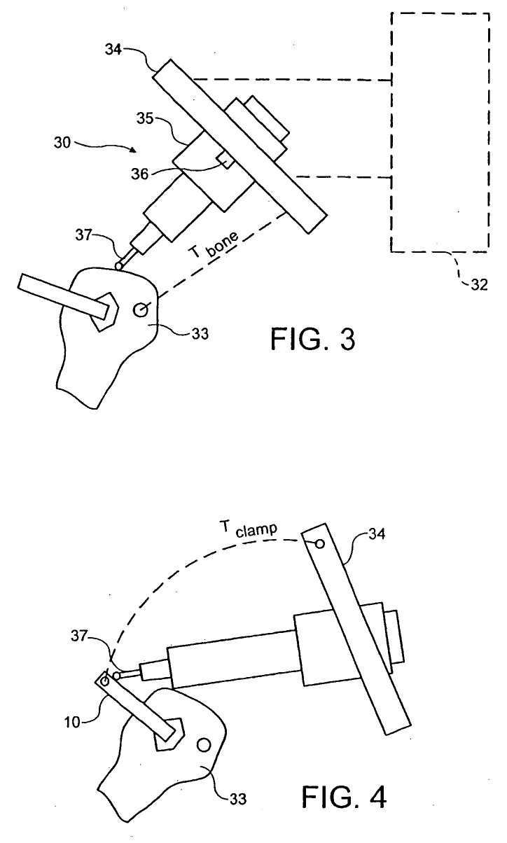

FIG. 3 shows a robot initially being registered to a bone;

FIG. 4 shows the robot being registered to the clamp of FIG. 1;

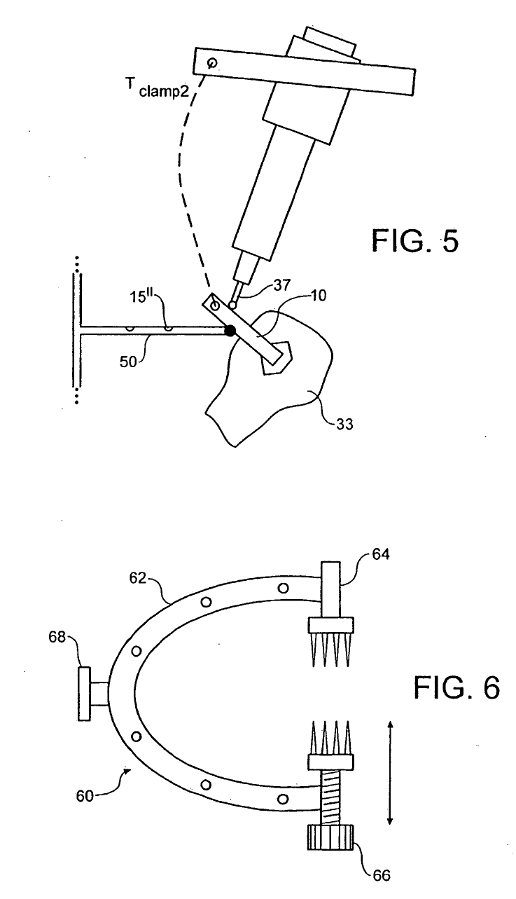

FIG. 5 shows the robot being re-registered to the clamp after the robot has been moved relative to the clamp and bone; and

FIG. 6 shows an alternative clamp in a second embodiment of the present invention.

The preferred embodiments of the present invention will now be described in connection with an exemplary application, namely the carrying out of a series of orthopaedic procedures.

First, the patient's bone is scanned using a three-dimensional scanning procedure such as computer tomography, and a three-dimensional surface model of the bone generated from the scan data. The patient is then prepared for surgery, the bone is exposed, and is clamped in a suitable position using a bone restraint system having one or more bone clamps 10 as shown in FIG. 1. The clamp 10 has a pair of jaws 12,14, each jaw terminating in bone-engaging spikes 20. The jaws are hinged at 16, and may be tightened by means of an adjustment screw 18. The clamp 10 forms part of or is held in place by a patient or bone restraint system 50 (not shown in FIG. 1 but illustrated very schematically in FIG. 5). Once the bone has been located as desired, the jaws 12,14 are closed, rigidly securing the clamp 10 to the bone, and the clamp is then locked in position with respect to the rest of the bone restraint system 50.

Surgery is carried out with the assistance of a small robot 30 (FIGS. 3 to 5) which is itself held in place at a desired location and orientation with respect to the clamped bone 33, by a gross positioning system 32 (shown schematically in FIG. 3). The robot 30 has a base 34 which is rigidly held in the desired position by the gross positioning system 32, and an operative portion 35 which may have a number of translational and rotational degrees of freedom with respect to the base 34, thereby allowing a cutting implement (not shown) to reach those areas of the bone 33 that need to be cut away. Sensors 36 measure the location of the robot operative portion 35 with respect to the base 34. As the size and configuration of the operative portion 35 is known, the sensors 36 therefore allow the position of any cutting implement or probe 37 on the operative portion to be accurately determined with respect to the base 34.

During any surgical procedure, the surgeon needs to know the precise orientation and location of the robot operative portion 35 (and hence any cutting implement attached to it) with respect to the digital model of the bone being held in computer memory. That is achieved, as shown in FIG. 3, by inserting a probe 37 into the end of the robot operative portion, and then touching that probe onto the exposed bone surface at a number of points. An iterative error-minimisation technique is then used to determine the relationship between the current true position of the bone, and hence the end of the probe, with the corresponding locations held in computer memory. The result is a transformation matrix T_{bone} which converts from the computer model reference frame (the frame in which the surgeon originally planned the procedures) to a real-world (robot referenced) frame in which those procedures will actually be carried out.

In this embodiment, the “robot frame” will be defined as that frame of reference in which the base 34 of the robot is held stationary by the gross positioner 32. As mentioned above, the position of the probe 37 (or of any cutting implement) within the robot frame may be determined by means of the operative portion sensors 36.

Once the robot has been registered to the bone surface, the first operative procedure may be undertaken. In a simple case, that may be all that is required, but the present invention is particularly applicable where there is a need to move the robot and/or to move the patient at the end of the first procedure, before putting the robot back and undertaking a second procedure.

In the preferred embodiment, prior to the start of the first procedure the robot is not only registered to the bone, as shown in FIG. 3, it is also registered to the clamp 10 as shown in FIG. 4. That is done, as shown in FIG. 2a, by locating a ball-head 20 of the probe 37 into a variety of holes or depressions 15 in an upper surface of the clamp. The depressions 15 are conical, with an outer diameter that is smaller than that of the ball-head 20. This accurately locates the centre of the ball-head with respect to the clamp, regardless of the angle 0 between the surface of the clamp and the length of the probe 37.

Depending upon the orientation of the clamp, some of the depressions 15 may not be accessible to the ball-head, as shown in FIG. 2b. In such a situation, alternate depressions 15′ in a side surface of the clamp may be used instead. Preferably, depressions are provided on each surface of both of the jaws 12,14 of the clamp so that the probe may be accurately positioned in at least some of the apertures regardless of clamp orientation. The depressions are preferably positioned such that from any direction at least four can be reached with the probe 37. This gives three points (the minimum required for three dimensional location) with a spare point for error correction through redundancy.

As shown in FIG. 4, prior to start of any operative procedures, the robot is registered to the clamp by touching the probe into at least four depressions, on both sides of the clamp. The positions of the holes are preferably non-equidistant so that, by touching the probe into two or perhaps three depressions on one of the jaws, the position of that jaw in space, relative to the robot base 34, is uniquely determined. Of course, because of the hinge 16, depressions on one jaw of the clamp will move relative to those of the other when the adjustment screw is tightened, and it is therefore desirable that the clamp tightness is not changed after the initial registration to the clamp has been completed. By touching sufficient depressions on both of the jaws of the clamp, it will be possible to determine, when re-registering later, whether there has been any relative movement between the jaws.

As shown in FIG. 4, the registration of the clamp to the robot provides an initial transformation matrix T_{clamp} which gives the clamp geometry in the robot reference frame. Conversely, its inverse T_{clamp}⁻¹ gives the robot position in terms of the clamp frame of reference.

Once T_{bone} and T_{clamp} have been determined, it is a mere mathematical exercise to map into the frame of reference used by the CT model the robot frame, the clamp frame and the bone frame. The location of the operative portion of the robot 35, and any cutting implement on it, may also be mapped into the same reference frame using the information from the sensors 36. By using the appropriate matrices, or their inverses, the surgery may be carried out in either the model reference frame, the bone reference frame, the clamp reference frame or the robot reference frame. For reasons given below, the clamp or bone reference frame is preferred.

Once both the bone and the clamp have been registered to the robot, in accordance with the preferred embodiment of the invention, any desired surgical procedure may then be carried out. At the end of that procedure, there may be a need to move the patient with respect to the gross positioner 32, or alternatively to move or remove the robot. Following completion of any further surgical procedures, the robot then needs to be put back into place (not necessarily in exactly the same position as before) so that some further computer-aided surgery procedures can be carried out. This requires, of course, that the new position of the robot should be known with respect to the bone.

As shown in FIG. 5, once the robot has been re-positioned, it is then re-registered to the clamp 10 by touching the probe 37 into at least four of the holes in the clamp. These do not have to be the original four holes, since the geometry of the clamp is known a priori. From the measured spacings and locations of these holes, the position of the clamp jaws can be determined, which allows a new transformation matrix T_{clamp2} to be calculated that relates the clamp frame to the robot base 34 (new co-ordinate reference frame). Since there has been no relative movement between the bone frame and the clamp frame, the matrix T_{clamp2} allows the bone frame to be transformed to the new robot frame, or vice-versa. Either can be transformed into the model reference frame since both the bone and the clamp will not have moved within that frame. The bone is still transformed from model co-ordinates to clamp co-ordinates using T_{bone} and T_{clamp}⁻¹ since that was the relationship between the bone and the robot during the initial surface registration. As before, further surgical procedures may be carried out, as desired, either in the model reference frame, the bone frame, the clamp frame, or the new robot frame. Preferably, the clamp or the bone frame is used since that allows the same co-ordinate reference frame to be used both before and after the repositioning of the robot.

It is within the knowledge of a skilled person to transform the reference frames, as desired, into the reference frame in which the operation is being carried out. Where the clamp frame is being used as the base frame, both the robot and the bone locations need to be transformed into that co-ordinate system, as follows:
T_{clamp}⁻¹=robot in clamp frame
T_{clamp}⁻¹T_{bone}=bone in clamp frame

After the robot has been positioned, the system calculates:
T_{clamp2}⁻¹=new robot position in clamp frame

In an alternative embodiment, the exact location of the depressions 15 within the clamp may not be known a priori to the system. Instead, after registration of the robot to the bone, the clamp position is “learned” by touching at least four depressions on the clamp, preferably on both jaws. After the robot has been moved, it is re-registered back to the clamp by touching those same depressions (or at least some of them) again. Provided that sufficient of the same depressions can be reached by the probe during re-registration, the precise location of the depressions on the clamp does not matter.

To ensure that the same depressions are re-visited during re-registration, each depression may be numbered and the corresponding number entered onto the computer system as the probe is touched against it. On re-registration, the computer system tells the surgeon which depressions have to be re-visited, and in which order.

Where the initial registration is used to learn the positions of the depressions, it is preferred that the “base frame” in which the surgical procedures are carried out is the robot frame. In such a case, the matrix T_{clamp} will simply be the identity matrix.

Instead of using depressions 15,15′ in the bone clamps 10, depressions 15″ could also be provided on the patient or bone restraint 50 (shown schematically in FIG. 5). Since the clamps 10 are fixedly secured to a frame of the patient restraint 50, the matrices T_{clamp} and T_{clamp2} could equally well be determined by touching the probe 37 into the depressions 15″ in the frame.

An alternative embodiment 60 of the clamp is shown in FIG. 6. A C-shaped clamp body 62 terminates at one end in a fixed spiked jaw 64 and at the other in a screwed, tightenable, jaw 66. A fastener 68 (present, but for clarity not shown in FIG. 1) is provided to allow securement of the clamp to a frame of a bone restraint 50 (FIG. 5).

The probe 37 used for registration is preferably inserted into a cutter chuck of the robot, in the position that would normally be occupied by the cutting mill. Because the cutter chuck has a definite end stop, the probe tip is at a definite location in relation to the geometry of the robot, as is the cutting mill during the cutting procedure. In an alternative arrangement (not shown) the probe need not be swapped with the cutter, but instead it may be inserted into a separate port at the end of the operative portion of the robot, for example adjacent to the cutter. In such a case, care must be taken to ensure that the geometry of the cutter does not foul access either to the bone or to any registration holes/depressions that the surgeon may require access to.

1. An apparatus for registering the position of a surgical robot prior to undertaking a surgical procedure, comprising a patient restraint including a plurality of marker locations thereon for receipt of a probe associated with a robot, the position of which is to be registered.

2. The apparatus as claimed in claim 1 in which the marker locations are on a bone clamp.

3. The apparatus as claimed in claim 1 in which the patient restraint comprises a bone clamp and a frame for holding the clamp, the marker locations being on the frame.

4. The apparatus as claimed in claim 1 in which the patient restraint comprises a bone clamp and a frame for holding the clamp, the marker locations being on the frame and on the clamp.

5. The apparatus as claimed in claim 2 in which the clamp has two relatively-movable jaws, each jaw having a plurality of marker locations.

6. The apparatus as claimed in claim 2 in which the marker locations are positioned so that at least three locations are accessible to a probe regardless of clamp orientation.

7. The apparatus as claimed in claim 1 in which the marker locations comprise holes or depressions in a surface of the patient restraint.

8. The apparatus as claimed in claim 7 in which the marker locations comprise conical depressions in a surface of the patient restraint.

9. The apparatus as claimed in claim 1 in which the marker locations are unequally spaced.

10. A system for registering the position of a surgical robot prior to undertaking a surgical procedure, the system comprising:
(a) a surgical robot;
(b) a probe associated with the robot; and
(c) a patient restraint including a plurality of marker locations thereon for receipt of the probe.

11. A system as claimed in claim 10 in which the marker locations are on a bone clamp.

12. A system as claimed in claim 10 in which the patient restraint comprises a bone clamp and a frame for holding the clamp, the marker locations being on the frame.

13. A system as claimed in claim 10 in which the patient restraint comprises a bone clamp and a frame for holding the clamp, the marker locations being on the frame and on the clamp.

14. A system as claimed in claim 11 in which the clamp has two relatively-movable jaws, each jaw having a plurality of marker locations.

15. A system as claimed in claim 11 in which the marker locations are positioned so that at least three locations are accessible to a probe regardless of clamp orientation.

16. A system as claimed in claim 10 in which the marker locations comprise holes or depressions in a surface of the patient restraint.

17. A system as claimed in claim 16 in which the marker locations comprise conical depressions in a surface of the patient restraint.

18. A system as claimed in claim 10 in which the marker locations are unequally spaced.

19. A system as claimed in claim 16 in which the probe has a ball-head with a diameter smaller than a diameter of the holes or depressions.

20. A system as claimed in claim 10 in which the probe is removable, and fits into a cutter chuck of the robot.

21. A system as claimed in claim 10 in which the probe extends adjacent to a cutter on the robot.

22. A system as claimed in claim 10, further including computer means for determining a transformation matrix T_{clamp} defining a patient restraint frame of reference with respect to a robot frame of reference, or vice versa.

23. A system as claimed in claim 22, further including computer means for determining a transformation matrix T_{bone} defining a patient frame of reference, as measured by touching the probe to a plurality of points on a body part of a patient, with respect to the robot frame of reference, or vice versa.

24. A system as claimed in claim 22, further including a computer means for determining a further transformation matrix T_{clamp2} defining a patient restraint frame of reference with respect to a new robot frame of reference, or vice versa.

25. A system as claimed in claim 10, further including computer memory means for holding a computer model of the marker locations, a cutter associated with the robot, and a patient body part on which a surgical procedure is to be undertaken.

26. A method of registering the position of a surgical robot prior to undertaking a surgical procedure, comprising the steps of:
(a) clamping a body part in a fixed position by means of a patient restraint; and
(b) registering a robot position by touching a probe associated with the robot to a plurality of marker locations on the patient restraint.

27. A method as claimed in claim 26 in which the body part is a bone.

28. A method as claimed in claim 26 in which the marker locations are individually identified, the method further including the step of touching selected markers and noting the identity of each selected marker.

29. A method as claimed in claim 26 or in which the marker locations are unequally spaced, the method further including the step of touching selected markers and automatically determining which individual markers have been touched by measuring the spacings between the selected markers.

30. A method as claimed in claim 26 further including the step of determining a transformation matrix T_{clamp} defining a patient restraint frame of reference with respect to a robot frame of reference, or vice versa.

31. A method as claimed in claim 30 further including the step of determining a transformation matrix T_{bone} defining a patient frame of reference, as measured by touching the probe to a plurality of points or a body part of a patient, with respect to the robot frame of reference, or vice versa.

32. A method as claimed in claim 30 further including the step of determining a further transformation matrix T_{clamp2} defining a patient restraint frame of reference with respect to a new robot frame of reference, or vice versa.

33. A method as claimed in claim 26 further including the step of generating a computer model including marker locations, the location of a cutter associated with the robot, and the patient body part.
